(19) United States
(12) Patent Application Publication
(10) Pub. No.: US 2017/0258629 A1
(43) Pub. Date: Sep. 14, 2017

(54) SEGMENTED LEG HEATER SYSTEM APPARATUS

(71) Applicant: Jacqueline Awasthi, Destrehan, LA (US)

(72) Inventor: Jacqueline Awasthi, Destrehan, LA (US)

(21) Appl. No.: 15/064,252

(22) Filed: Mar. 8, 2016

(51) Int. Cl. A61F 7/00

(52) U.S. Cl. CPC A61F 7/007; A61F 2007/0039; A61F 2007/0029; A61F 2007/0071; A61F 2007/0086; A61F 2007/0095; A61F 2007/0078; A61F 2007/0082

(57) ABSTRACT

A segmented limb heater system apparatus for controlled therapeutic heating of a limb having different segments and joints, differentially heating different segments or joints of a limb, with safe, automatic controls based on sensing of the different actual temperatures, in a portable, easy to use embodiment.

### BACKGROUND OF THE INVENTION

The present invention provides a segmented limb heater system apparatus for controlled therapeutic heating of a limb having different segments and joints, differentially heating different segments or joints of a limb, with safe, automatic controls based on sensing of the different actual temperatures, in a portable, easy to use embodiment.

Therapeutic heating of parts of the body is an effective form of pain management. Application of heat causes blood vessels in the heated area to dilate, which enhances perfusion to the heated tissue. A heating pad is used to apply such therapeutic heating. Most heating pads can be used at home or otherwise outside of a health-care facility, but such use is limited by the availability of a source of heat or a source of electricity to produce such heat. This presents a difficulty in using a heating pad while driving or otherwise traveling, when attending a public event, concert, or game, and generally when away from the home or office.

The improper application of heat, such as too much heat for too long a duration, can be harmful. In a health-care facility, a health-care personnel must monitor therapeutic heating to avoid overexposure, often for more than one patient on different therapeutic regimes. At home, the patient or the caregiver must monitor the therapeutic heating, and the patient might fall asleep or otherwise lose attention, or might perceive a need for more heat than is prescribed.

When applying therapeutic heat to a limb, such as an arm or a leg, there are considerations of whether all or just some segments or joints need to be treated, and whether each needs the same or different intensities and durations of heat. Even where an equal temperature along the entire limb is wanted, the different segments and joints of a limb, with different shapes and different ratios of flesh and bone, absorb, retain, and dissipate heat in different ways, and a single heating pad with a single setting is not well suited to fully covering and differentially heating the segments and joints of a limb. And such a single heating pad is susceptible to overheating portions of the limb when it is other portions of the limb that are being monitored for heat. For example, a person's foot might always feel cold, and a low level of heat for a long duration might be appropriate or might be what the person choses to apply in that particularly situation. But the heat applied to the knee, which might be the part needing the heat therapy, might be of too much or too little intensity or duration where the single point of control is set with reference to the foot.

Known heating pads are regulated with regard to the heat produced by the pad, not to the actual heat of the target body part. But variations in the configuration and makeup of the body part, and variations in the fitting of the heating pad, affect the effective absorption, retention, and dissipation of heat, which is not accounted for where the heating pad is only monitoring its own heat.

There is a need for a system to differentially heat different segments or joints of a limb, with safe, automatic controls based on sensing of the different actual temperatures, in a portable, easy to use embodiment.

The references disclose the use of a heated pad in conjunction with an encasement that applies the heat to various parts of the body, whether for therapeutic purposes or for protection from the external cold.

U.S. Pat. No. 1,358,509, issued Nov. 9, 1920 to Benjamin Birkenfeld on an “Electrically Heated Garment,” relates to an electrically heated garment, designed to be applied as an outer garment or protective covering whereby to impart to the wearer sufficient warmth to withstand cold and inclement weather. The Birkenfeld garment covers an electrically heated garment comprising a body section and a limb section having associated therewith a series of heating coils, means whereby electric current may pass through the heating coils of the body section and limb section simultaneously, and means for cutting out the current from one or both of said sections as desired, without removing the sections.

U.S. Pat. No. 3,623,485, issued Nov. 30, 1971 to David Bowen Price for a “Heating Pad Cover,” relates to a fabric covering for a therapeutic apparatus, and more particularly to a fabric cover for a heating pad on the body of the wearer that provides variable adjustment. The fabric cover for a heating pad has an exposed surface of the cover comprising a fabric with a nap of loose closed loop, nonmatting plush pile, and a strap or extension of the cover attached at one end of the cover. A strip of Velcro hook material is secured at the other end thereof, on the side opposite the plush pile surface of the cover, so that when the cover, enclosing the pad, and the extension are wrapped about a person, the Velcro hook will come in contact with the plush pile and secure itself thereto to hold the pad about the wearer and provide for adjustable attachment.

U.S. Pat. No. 4,404,460 issued Sep. 13, 1983 to John F. Kerr on “Controllably Heated Clothing.” The Kerr invention relates generally to heated clothing, and more particularly concerns a heated suit system that is modular and enables selective wearing of certain sections of the suit without impairment of the heating function. The heated clothing includes (1) multiple clothing sections adapted to cover different portions of the human body, (2) electrical wires carried by such sections, including bus wires and heater wires, and (3) circuitry for controlling battery-powered electrical current flow to such wires, and including switch structure connected in series with one or more of the heater wires for interrupting current flow thereto without requiring disconnection of bus wires.

U.S. Pat. No. 5,032,705 issued Jul. 16, 1991 to Barry D. Batcheller et al. for an “Electrically Heated Garment.” The electrically heated, cold-weather garment includes a lightweight, stretchable, form-fitting fabric for covering portions of the body of a wearer of the garment; a plurality of flexible, electrical heating wires cover-stitched to the fabric by sewing; an electronic controller for controlling current flowing through each of the heating wires in a pulse-width modulated fashion, to thereby independently control the heat generated by each heating wire; a plurality of potentiometers for controlling the level of power supplied to each heating wire; and a master power level potentiometer for controlling the power supplied to each of the heating wires in a uniform and simultaneous fashion. In a first preferred embodiment of the Batcheller garment, the controller utilizes a combination of analog and digital-like signals to control in a pulse-width modulated fashion the current flow through the heating elements. In a second preferred embodiment, the controller includes a microprocessor which is operable to sense changes in the temperature of the heating wires themselves, and to regulate automatically and independently the power supplied to each of the heating wires.

U.S. Pat. No. 3,634,655, issued Jan. 11, 1972 to Mickey S. Jordan, covers a “Multiple Heating Pad Assembly.” The heating pad unit comprises a pair of individual heating pads which may be selectively joined together and positioned one over the other to form a single twin-heat unit. The individual heating pads may also be separated and used as two heating pads for positioning against different parts of a patient's body. A principal object of the Jordan invention is to provide an improved heating pad assembly which is selectively adjustable to be used either as a single twin-heat unit or as a pair of separate, individual heating pads positioned against different parts of the body at the same time. The heating pad assembly comprises a pair of flat heating pads similar in size and shape, electrical heating wires in each heating pad, a slide fastener connected to the mutual edges of both of said heating pads and arranged to fasten said heating pads at several of their mutual edges in overlapping relationship, or to fasten said heating pads along an adjacent edge of each heating pad in an open flat spread relationship, or to release the heating pads from each other, and electrical extension cord connected to said electrical heating wires and extending from each heating pad, a single heat control device connected to each of the electrical extension cords, an electrical power cord connected to said heat control device for connection to a source of electrical power, the heat control device being constructed and arranged to individually control the heat intensity emitted from each of the heating pads, with the heat control device including means for retracting the electrical power cord.

U.S. Publication No. 2013/0006335 was published Jan. 3, 2013 by Mark H. Lowe on a “Thermal Therapy System.” The thermal therapy system provides thermal treatment to a body requiring treatment. The disclosed system may include a plurality of therapy wraps, each configured to exchange heat with the body. The therapy wraps may be secured to different locations on a substrate and unfurled onto a rigid board for supporting the body. A control unit may be configured to independently control the therapy wraps. The therapy wraps may have a plurality of layers. One of the layers is a heat exchange layer comprising a heat transfer device for exchanging heat with the body. One of the layers may be a compressive layer for applying a compressive force to the body. One of the layers may be a structural layer including a rigid structural member. Also disclosed in the Lowe publication are methods of administering a temperature-controlled treatment to an animate body.

Korean Publication No. 2010002170 was published Mar. 4, 2010 by Kim G. and Song S. on a “Heating Knee Pad.” The heated knee pad has a band of elasticity cover formed on the top part or lower part of the pad, and a hook and loop fastener tape sewn to attach and detach from the cover in the backside surface of the bag. The pad features a recharagable battery in which a terminal connection outlet is molded in one side, and a bag formed so as to open and close a side cover in a state adhered to a front side of an elastic cover. A connection terminal is formed at an end of a wire that is connected in the state adhered to the backside surface of the cover. A band of the elastic cover is formed to a top part or lower part of the pad. Velcro tape is sewn to attach and detach from the cover on the backside surface of the bag. The system provides functions of maintenance of heat and movement of the knee, conveniently displayed through a swollen joint curved surface. The bag for placing the rechargeable battery may also be attached and detached as desired.

Chinese Publication No. 204599387 was published on Sep. 2, 2015 by Song C., Song X., Zhang T. and Zhao J., and covers a mobile phone-based wireless charging controlled knee heating pad device for use by, for example, traffic police. The pad has a charging battery that is connected with a wireless charging receiver by a standard micro USB interface. The heating pad comprises a charge launch module and mobile charge module, and uses a novel carbon fiber heating material, wherein warmth and a winter-proofing effect a provided to the user. The infrared heat radiation enhances lymph circulation, promotes blood circulation by removing blood stasis, and promotes metabolism. The system also provides for a safer heating system by providing a protective circuit. The mobile phone software provides settings of the heating pad and a monitoring interface, which displays the present settings, knee temperature, voltage, and electric current parameters.

### SUMMARY OF THE INVENTION

The present invention is a segmented limb heater system apparatus for controlled therapeutic heating of a limb having different segments and joints, differentially heating different segments or joints of a limb, with safe, automatic controls based on sensing of the different actual temperatures, in a portable, easy to use embodiment.

### BRIEF DESCRIPTION OF DRAWINGS

Reference will now be made to the drawings, wherein like parts are designated by like numerals, and wherein:

FIG. 1 is a schematic view of the segmented limb heater system in use on a person's leg; and

FIG. 2 is a schematic view of the segmented limb heater system in use on a horse's leg.

### DETAILED DESCRIPTION OF THE INVENTION

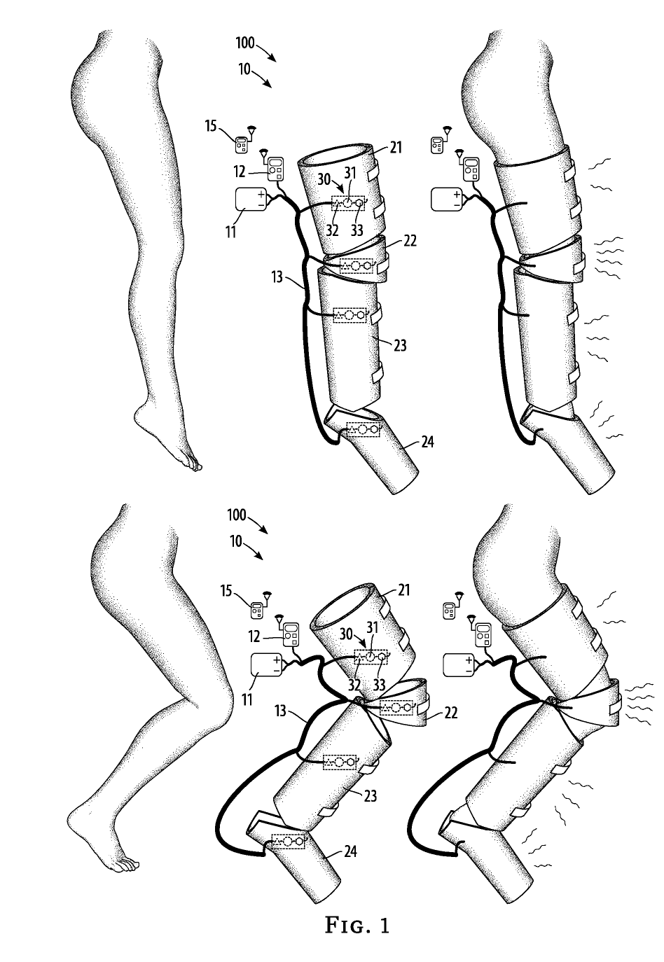

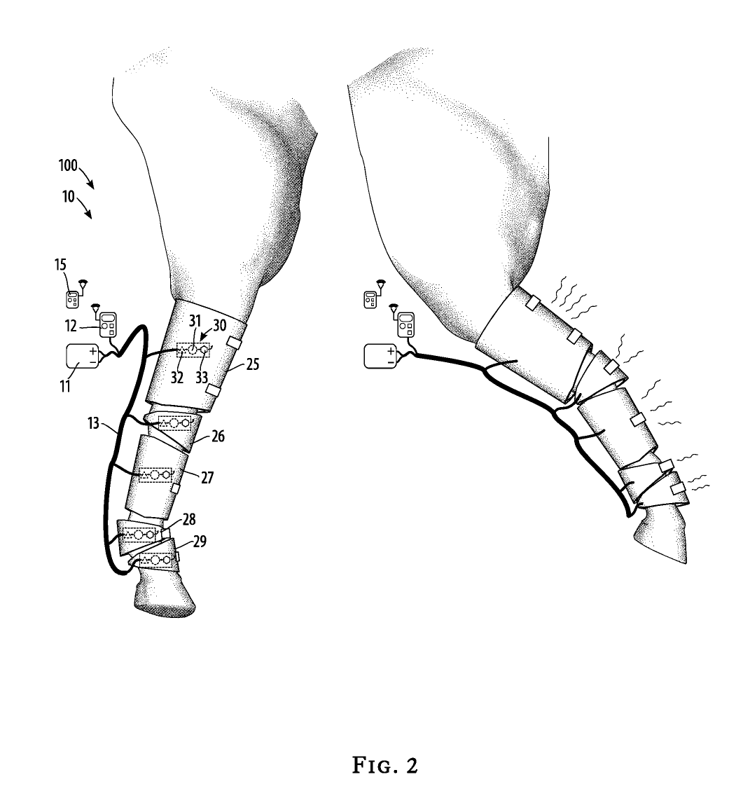

Referring to FIG. 1 & FIG. 2, the segmented limb heater system method 100 and apparatus 10 are shown schematically in use on the legs of a person and a horse. The invention is for use on other segmented limbs, such as arms, of a variety of beings, and can be worn directly against the flesh or over clothing or bandages, as appropriate.

In overview, electric power, preferably at low voltage for safety and efficiency, is supplied by a power source 11 over a power and control bus 13, for each of several heating segments 21-29 to generate heat, all under control of a control unit 12 which monitors and implements an appropriate schedule of heating events, with regard to heat intensity and time of application, individually for all of the segments.

Although higher voltage alternating current or AC could it be used in this invention, doing so would introduce a much higher risk of painful or fatal electrocution in the event of any system failure, and therefore would require more complex and expensive safety features. And AC power would provide no significant offsetting advantage here, and would be difficult to implement in a portable system.

The individual heating segments 21-29 are made out of at least moderately flexible fabric or sheeting having embedded electrical resistance heating elements, such as heater wires, within, and a heat-dispersing cover layer or separate cover to prevent burns caused by direct or under-modulated contact of heating elements and flesh.

The most useful embodiments of individual heating segments 21-29 are likely to be flexible pads which can be wrapped around the limb segment and adjustably fastened by known means such as hook and loop strips, snaps, or magnetic closures.

Each individual heating segment 21-29 contains an individual segment controller 30 that controls the application of heat by that segment. Although in normal use the intensity and duration of heating in one segment or another is under the central control of the control unit 12, the invention provides two fail-safe devices associated with each individual segment controller 30, an overheat disconnect 32 and an overtime disconnect 33, which will prevent too much heat or too much heating time being applied by that segment no matter what larger system malfunction or maladjustment might be directing otherwise. Each individual segment controller 30 is also associated with an isolating thermal sensor 31, designed to correctly sense the heat in each limb segment without interference caused by sensing the heat of the heating segment itself. This isolation can be achieved through a combination of the design and the placement of the isolating thermal sensor 31. In use, the heat generated by any particular heating segment might be all efficiently transferred into the subject's limb segment or might be partially dissipated because of configuration or fitting. The use of isolating thermal sensors 31 ensures that the actual therapeutic heat applied to each limb segment is known and accounted for.

The control unit 12 has bi-directional communication with each individual heating segment 21-29 through the power and control bus 13. Such communication can be implemented over dedicated wires separate for each heating segment, over one dedicated pair of wires used by all heating segments, or over the low-voltage DC wires used for power transmission. Communication over power wires is accomplished by injecting and filtering a separate signal. Communication over common wires is accomplished either by signaling at different frequencies or by incorporating identifying information into the signal itself. The control unit 12 receives from the several isolating thermal sensors 31 updated information about the actual temperature applied to each limb segment. The control unit 12 can be programmed to conduct a heat-therapy session with the amount of heat applied and the time duration of application for each individual limb segment controlled under a desired therapeutic regime. Such a therapeutic regime might call for all limb segments to be maintained at the same temperature, or might call for different temperatures for different limb segments. The control unit 12 can activate and deactivate the heating elements in the various individual heating segments 21-29 in order to achieve and maintain the desired therapeutic temperature for the desired amount of time.

Optionally, a remote unit 15 can be provided to interface with the control unit 12 and provide an additional means of controlling and monitoring the heat therapy provided by the invention. Such a remote unit 15 can communicate wirelessly with the control unit. Such a remote unit might take the form of a small handheld remote that can be carried by a companion or caregiver, or might take the form of a centralized remote control and monitoring unit for a healthcare facility managing a significant number of heat-therapy subjects.

The individual heating segments 21-29 are configured such that they provide proper coverage and heating to the relevant segment of the limb, without restricting the movement of the limb unreasonably. For a hinge or pivot joint, the heating segment 22, 24, 26, 28, 29, can be of a generally wedged shape, as shown, when in use. Other heating segments can be slightly cut away at their ends, as shown, in order to provide additional space for a hinge or pivot joint. A standard configuration of heating segments 21-29, such as for an adult person's leg, as shown, can be made up into a single garment by making flexible attachments of the segments or by attaching the segments to a single flexible garment, such as a stocking. But the separate heating segments provide for a more easy putting on and taking off, especially for an immobilized person, and combination into a single garment might be disadvantageous in some circumstances.

The power source 11 can provide electric storage, such as a battery or capacitor, or can provide an electric transformer to convert higher AC voltage to lower DC voltage, or can provide both. Having a battery provides portability and allows heat therapy to be administered away from an electrical outlet, such as when driving or riding, working, or pursuing other activities. Because the heating segments are connected in parallel through the power and control bus 13, the same low DC voltage is equally available to each segment. The heating process draws considerable amperage from the power source 11, and a battery providing significant amp hours is needed. For battery operation, in order to avoid too much simultaneous draw, the control unit 12 can be programmed to ensure that not more than one or not more than two heating segments are actively heating at the same time. Because the heat from the heating wires or heating elements is intended to disburse slowly, and is retained within the closed heating segment and retained by the flesh being heated, the control unit 12 can maintain an essentially constant temperature in several segments by pulsing or actively heating segments for relatively short times and in a relatively fast rotation among the segments. Such a pulsing or rotating pattern avoids the drawing of too much amperage because only one heating segment is drawing current at any given time, and no segment is drawing excess or unneeded current when the subject limb segment is already at proper temperature. This capacity to manage and limit the draw on a battery, provided by the invention, allows heat therapy to be administered or self-administered in locations and under circumstances that would be cumbersome if an electrical outlet or a much larger battery were needed.

Many changes and modifications can be made in the present invention without departing from the spirit thereof. I therefore pray that my rights to the present invention be limited only by the scope of the appended claims.

I claim:

1. A segmented limb heater system apparatus for controlled therapeutic heating of a limb having different segments and joints, comprising:
(i) more than one individual heating segment adapted to fit on or around a particular segment or joint of the limb and to provide electrical-resistance heat essentially evenly distributed within said individual heating segment upon activation of said individual heating segment;
(ii) more than one individual segment controller arrayed one-for-one upon or within said individual heating segments, adapted to control the activation of the associated individual heating segment, and further comprising an isolating thermal sensor adapted to sense the temperature of the limb segment or joint without false sensing of the heat emanating from said individual heating segment;
(iii) a power and control bus adapted to convey heating power to said individual heating segments and to convey temperature information from said individual segment controllers;
(iv) a power source adapted to provide heating power to said individual heating segments via said power and control bus; and
(v) a control unit adapted to activate and deactivate said individual heating segments as needed to provide a desired therapeutic regime, based on temperature readings from said isolating thermal sensors received on said power and control bus, and activation signals sent from said control unit to said individual heating segments on said power and control bus;
where said control unit is programmed to provide a therapeutic regime of a desired amount of therapeutic heat for a desired amount of time specific to each limb segment or joint, where said isolating thermal sensors monitor the heat of each limb segment or joint, and where said control unit activates and deactivates each said individual heating segment as needed to realize the therapeutic regime.

2. The segmented limb heater system apparatus of claim 1, where said individual segment controller further comprises an overheat disconnect adapted to deactivate said individual heating segment upon its reaching a defined safe threshold temperature.

3. The segmented limb heater system apparatus of claim 1, where said individual segment controller further comprises an overtime disconnect adapted to deactivate said individual heating segment upon its being activated for a defined safe threshold amount of time.

4. The segmented limb heater system apparatus of claim 1, further comprising a remote unit adapted to interface with said control unit.

5. The segmented limb heater system apparatus of claim 1, where said power source provides low-voltage direct-current electrical heating power over said power and control bus.

6. The segmented limb heater system apparatus of claim 1, where said power source further comprises an electrical storage battery.

7. The segmented limb heater system apparatus of claim 1, where said power source further comprises an AC-to-low-voltage-DC transformer.

8. The segmented limb heater system apparatus of claim 1, where said control unit activates and deactivates said individual heating segments in a pattern that ensures that not more than a set threshold number of individual heating segments are active and drawing current at any one time.

9. The segmented limb heater system apparatus of claim 1, where said control unit activates and deactivates said individual heating segments in a pattern that ensures that only one individual heating segment is active and drawing current at any one time.

10. The segmented limb heater system apparatus of claim 1, where said individual heater segments are shaped such that movement of the limb segments and joints is allowed.
